(19) United States
(12) Patent Application Publication
(10) Pub. No.: US 2023/0317300 A1
(43) Pub. Date: Oct. 5, 2023

(54) DETECTING IBD EFFICIENTLY USING A DISTRIBUTED SYSTEM

(71) Applicant: Ancestry.com DNA, LLC, Lehi, UT (US)

(72) Inventor: Keith D. Noto, San Francisco, CA (US)

(21) Appl. No.: 18/057,700

(22) Filed: Nov. 21, 2022

Related U.S. Application Data
(60) Provisional application No. 63/326,392, filed on Apr. 1, 2022.

(51) Int. Cl. G16H 70/60

(52) U.S. Cl. CPC G16H 70/60

(57) ABSTRACT

Disclosed herein relates to a method that uses the RAM of multiple servers to increase the efficiency of identifying segments of a target dataset that match segments of other datasets in a database. An encoding system may encode large genetic datasets to produce pairs of bitmap sequence pairs that correspond to an encoding scheme. The servers each store portions of the database in their hard drives based on a shared characteristic of the genetic datasets in the database, such as ethnicity or location of birth. The servers encode data from their hard drives and sustain the encoded data in their RAM. A target, or query, individual is input for matching. The servers match the encoded data of the target individual with encoded data in their flash drives and can determine a relationship. The servers sustain the encoded data in RAM to compare against subsequent target individuals.

### CROSS-REFERENCE TO RELATED APPLICATION

This application claims priority to U.S. Provisional Application No. 63/326,392, filed on Apr. 1, 2022, which is incorporated by reference herein for all purposes.

### BACKGROUND

A large-scale database such as a genealogy database can include billions of data records. This type of database may allow users to build family trees, research their family history, and make meaningful discoveries about the lives of their ancestors. Users may try to identify relatives with datasets in the database. However, identifying relatives in the sheer amount of data is not a trivial task. Datasets associated with different individuals may not be connected without a proper determination of how the datasets are related. Comparing a large number of datasets without a concrete strategy may also be computationally infeasible because each dataset may also include a large number of data bits. Given an individual dataset and a database with datasets that are potentially related to the individual dataset, it is often challenging to identify a dataset in the database that is associated with the individual dataset.

The genetic data for even a single individual can be quite large. Matching segments that are almost always not a perfect match adds complexity to the process. Running an effective algorithm that can quickly identify matches among the individual datasets in a large-scale database can help make the process more efficient. However, having to re-encode large genetic datasets every time the matching algorithm runs is extremely time consuming and inefficient.

### SUMMARY

Disclosed herein relates to example embodiments that identify one or more segments of a target dataset that match segments of other datasets in a database and sustain encoded genetic datasets in random-access memory (RAM) of multiple servers. In some embodiments, a computer-implemented method stores genetic datasets of multiple individuals on one or more hard drives of a database. The genetic datasets may be encoded. The encoding scheme generates pairs of encoded bitmap sequences. The encoding scheme defines a sequence of values based on homozygosity of the genetic dataset of each individual. The method also includes storing the pairs of bitmap sequences in RAM of a plurality of servers. Each server's RAM stores the pairs of encoded bitmap sequences of a portion of a large dataset of genetic data. The portions of encoded bitmap sequences in each server do not overlap, according to some embodiments. RAM is volatile and has faster processing speed than the one or more hard drives of the servers. The computer-implemented program receives an input pair of encoded bitmap sequences for a target, or query, individual. The program uses the input pair of encoded bitmap sequences for a target individual to determine relationships between the target individual and the plurality of individuals whose encoded genetic data are stored in the RAM. The program determines matched segments between the target individual and the plurality of individuals. To determine matched segments, the program compares the input pair of the target individual to the pairs stored in the RAM. Each server operates in parallel with the other servers. The computer-implemented program computes, for each server, a relationship between the target individual and an individual of the plurality of individuals. The program collates the computed relationships. The servers sustain the pairs of encoded bitmap sequences of the plurality of individuals in RAM, to be used again when another target individual is loaded.

In yet another embodiment, a non-transitory computer-readable medium that is configured to store instructions is described. The instructions, when executed by one or more processors, cause the one or more processors to perform a process that includes steps described in the above computer-implemented methods or described in any embodiments of this disclosure. In yet another embodiment, a system may include one or more processors and a storage medium that is configured to store instructions. The instructions, when executed by one or more processors, cause the one or more processors to perform a process that includes steps described in the above computer-implemented methods or described in any embodiments of this disclosure.

### BRIEF DESCRIPTION OF THE DRAWINGS

Figure (FIG. 1 illustrates a diagram of a system environment of an example computing system, in accordance with some embodiments.

FIG. 2 is a block diagram of an architecture of an example computing system, in accordance with some embodiments.

FIG. 3 is a flowchart depicting an example process for determining relationships and sustaining encoded bitmap sequences, in accordance with some embodiments.

FIG. 4 is a depiction of an example process for determining identity by descent (IBD) matches between a target individual and multiple individuals, in accordance with some embodiments.

FIG. 5 is a conceptual diagram illustrating an example encoding scheme that may be used to encode a genetic dataset to generate a pair of encoded bitmap sequences, in accordance with some embodiments.

FIG. 6 is a conceptual diagram illustrating an example sampling process for turning a pair of bitmap sequences into a pair of sparse bitmap sequences, in accordance with some embodiments.

FIG. 7A is a block diagram illustrating the architecture of a distributed computing system that may be used to carry out the IBD estimation process.

FIG. 7B is a block diagram illustrating the multiple servers used in the distributed computing system for carrying out the IBD estimation process, while sustaining encoded, processed data in random-access memory (RAM) of each server.

FIG. 8 is a block diagram of an example computing device, in accordance with some embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

The figures (FIGs.) and the following description relate to preferred embodiments by way of illustration only. One of skill in the art may recognize alternative embodiments of the structures and methods disclosed herein as viable alternatives that may be employed without departing from the principles of what is disclosed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Configuration Overview

In some embodiments, a system architecture is described to increase the efficiency of determining relationships between a target individual and a plurality of individuals. An encoding scheme is used to encode the homozygous locations of different types of data values. The encoding scheme may output pairs of bitmap sequences, each encoding the homozygous locations of different types of data values. For example, the first bitmap sequence of the pair carries information on the locations of a first type of data values (e.g. major alleles) of an individual's dataset. The second bitmap sequence of the pair carries information on the locations of a second type of data values (e.g., minor alleles) of the individual's dataset.

The system determines matches between a target individual and a plurality of individuals by comparing the target individual's bitmap pairs to those of a representative individual stored in RAM of the servers. Each server may store a portion of a larger genetic database, portioned out based on a shared quality between the individuals in the group. The representative individual's encoded genotype is stored in the RAM for comparison. The pairs of encoded bitmap sequences are sustained in the RAM of a plurality of servers rather than being re-encoded and re-loaded for each comparison. The encoded bitmap sequences may be sustained in the RAM for a user-defined period of time or until the servers are powered off. The servers may run the bitmap matching in parallel, increasing the efficiency of the matching process. The encoded datasets may be stored in hash tables containing a hash of a user's name, account name, date of birth, and other contextual information. The computer-implemented method may receive a second input pair of encoded bitmap sequences corresponding to a second target individual. The second input pair of bitmap sequences is then compared to the pairs of encoded bitmap sequences that are sustained in the RAM of the servers. The system may continue for a plurality of target individuals, without re-encoding genotypes from genetic datasets stored on one or more hard drives.

### Example System Environment

FIG. 1 illustrates a diagram of a system environment 100 of an example computing server 130, in accordance with some embodiments. The system environment 100 shown in FIG. 1 includes one or more client devices 110, a network 120, a genetic data extraction service server 125, and a computing server 130. In various embodiments, the system environment 100 may include fewer or additional components. The system environment 100 may also include different components.

The client devices 110 are one or more computing devices capable of receiving user input as well as transmitting and/or receiving data via a network 120. Example computing devices include desktop computers, laptop computers, personal digital assistants (PDAs), smartphones, tablets, wearable electronic devices (e.g., smartwatches), smart household appliances (e.g., smart televisions, smart speakers, smart home hubs), Internet of Things (IoT) devices or other suitable electronic devices. A client device 110 communicates to other components via the network 120. Users may be customers of the computing server 130 or any individuals who access the system of the computing server 130, such as an online website or a mobile application. In some embodiments, a client device 110 executes an application that launches a graphical user interface (GUI) for a user of the client device 110 to interact with the computing server 130. The GUI may be an example of a user interface 115. A client device 110 may also execute a web browser application to enable interactions between the client device 110 and the computing server 130 via the network 120. In another embodiment, the user interface 115 may take the form of a software application published by the computing server 130 and installed on the user device 110. In yet another embodiment, a client device 110 interacts with the computing server 130 through an application programming interface (API) running on a native operating system of the client device 110, such as IOS or ANDROID.

The network 120 provides connections to the components of the system environment 100 through one or more sub-networks, which may include any combination of local area and/or wide area networks, using both wired and/or wireless communication systems. In some embodiments, a network 120 uses standard communications technologies and/or protocols. For example, a network 120 may include communication links using technologies such as Ethernet, 802.11, worldwide interoperability for microwave access (WiMAX), 3G, 4G, Long Term Evolution (LTE), 5G, code division multiple access (CDMA), digital subscriber line (DSL), etc. Examples of network protocols used for communicating via the network 120 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over a network 120 may be represented using any suitable format, such as hypertext markup language (HTML) or extensible markup language (XML). In some embodiments, all or some of the communication links of a network 120 may be encrypted using any suitable technique or techniques such as secure sockets layer (SSL), transport layer security (TLS), virtual private networks (VPNs), Internet Protocol security (IPsec), etc. The network 120 also includes links and packet switching networks such as the Internet.

Individuals, who may be customers of a company operating the computing server 130, provide biological samples for analysis of their genetic data. Individuals may also be referred to as users. In some embodiments, an individual uses a sample collection kit to provide a biological sample (e.g., saliva, blood, hair, tissue) from which genetic data is extracted and determined according to nucleotide processing techniques such as amplification and sequencing. Amplification may include using polymerase chain reaction (PCR) to amplify segments of nucleotide samples. Sequencing may include sequencing of deoxyribonucleic acid (DNA) sequencing, ribonucleic acid (RNA) sequencing, etc. Suitable sequencing techniques may include Sanger sequencing and massively parallel sequencing such as various next-generation sequencing (NGS) techniques including whole genome sequencing, pyrosequencing, sequencing by synthesis, sequencing by ligation, and ion semiconductor sequencing. In some embodiments, a set of SNPs (e.g., 300,000) that are shared between different array platforms (e.g., Illumina OmniExpress Platform and Illumina HumanHap 650Y Platform) may be obtained as genetic data. Genetic data extraction service server 125 receives biological samples from users of the computing server 130. The genetic data extraction service server 125 performs sequencing of the biological samples and determines the base pair sequences of the individuals. The genetic data extraction service server 125 generates the genetic data of the individuals based on the sequencing results. The genetic data may include data sequenced from DNA or RNA and may include base pairs from coding and/or noncoding regions of DNA.

The genetic data may take different forms and include information regarding various biomarkers of an individual. For example, in some embodiments, the genetic data may be the base pair sequence of an individual. The base pair sequence may include the whole genome or a part of the genome such as certain genetic loci of interest. In another embodiment, the genetic data extraction service server 125 may determine genotypes from sequencing results, for example by identifying genotype values of single nucleotide polymorphisms (SNPs) present within the DNA. The results in this example may include a sequence of genotypes corresponding to various SNP sites. A SNP site may also be referred to as a SNP loci. A genetic locus is a segment of a genetic sequence. A locus can be a single site or a longer stretch. The segment can be a single base long or multiple bases long. In some embodiments, the genetic data extraction service server 125 may perform data pre-processing of the genetic data to convert raw sequences of base pairs to sequences of genotypes at target SNP sites. Since a typical human genome may differ from a reference human genome at only several million SNP sites (as opposed to billions of base pairs in the whole genome), the genetic data extraction service server 125 may extract only the genotypes at a set of target SNP sites and transmit the extracted data to the computing server 130 as the genetic dataset of an individual. SNPs, base pair sequence, genotype, haplotype, RNA sequences, protein sequences, and phenotypes are examples of biomarkers.

The computing server 130 performs various analyses of the genetic data, genealogy data, and users' survey responses to generate results regarding the phenotypes and genealogy of users of computing server 130. Depending on the embodiments, the computing server 130 may also be referred to as an online server, a personal genetic service server, a genealogy server, a family tree building server, and/or a social networking system. The computing server 130 receives genetic data from the genetic data extraction service server 125 and stores the genetic data in the data store of the computing server 130. The computing server 130 may analyze the data to generate results regarding the genetics or genealogy of users. The results regarding the genetics or genealogy of users may include the ethnicity compositions of users, paternal and maternal genetic analysis, identification or suggestion of potential family relatives, ancestor information, analyses of DNA data, potential or identified traits such as phenotypes of users (e.g., diseases, appearance traits, other genetic characteristics, and other non-genetic characteristics including social characteristics), etc. The computing server 130 may present or cause the user interface 115 to present the results to the users through a GUI displayed at the client device 110. The results may include graphical elements, textual information, data, charts, and other elements such as family trees.

In some embodiments, the computing server 130 also allows various users to create one or more genealogical profiles of the user. The genealogical profile may include a list of individuals (e.g., ancestors, relatives, friends, and other people of interest) who are added or selected by the user or suggested by the computing server 130 based on the genealogical records and/or genetic records. The user interface 115 controlled by or in communication with the computing server 130 may display the individuals in a list or as a family tree such as in the form of a pedigree chart. In some embodiments, subject to user's privacy setting and authorization, the computing server 130 may allow information generated from the user's genetic dataset to be linked to the user profile and to one or more of the family trees. The users may also authorize the computing server 130 to analyze their genetic dataset and allow their profiles to be discovered by other users.

### Example Computing Server Architecture

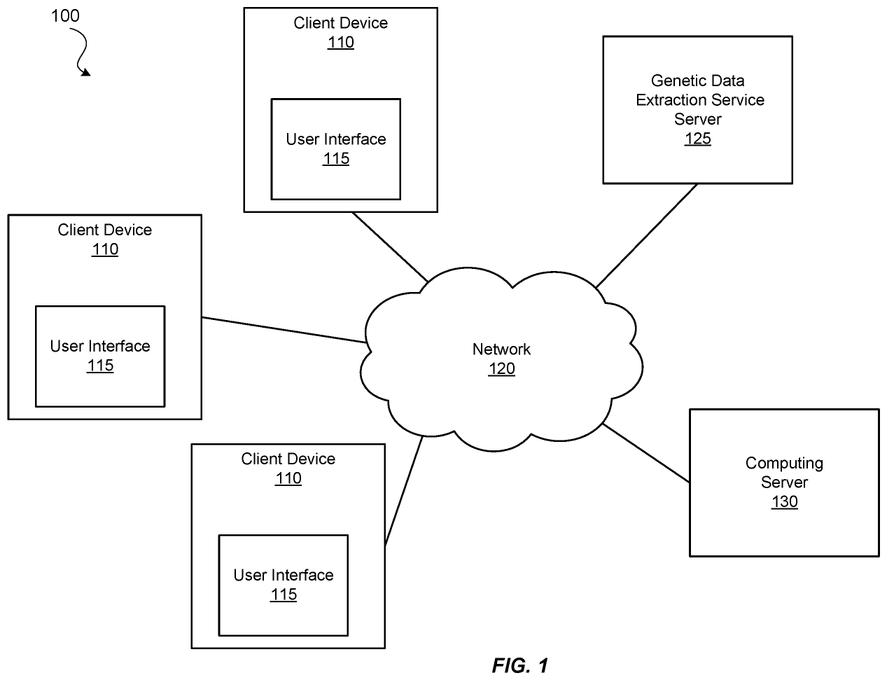

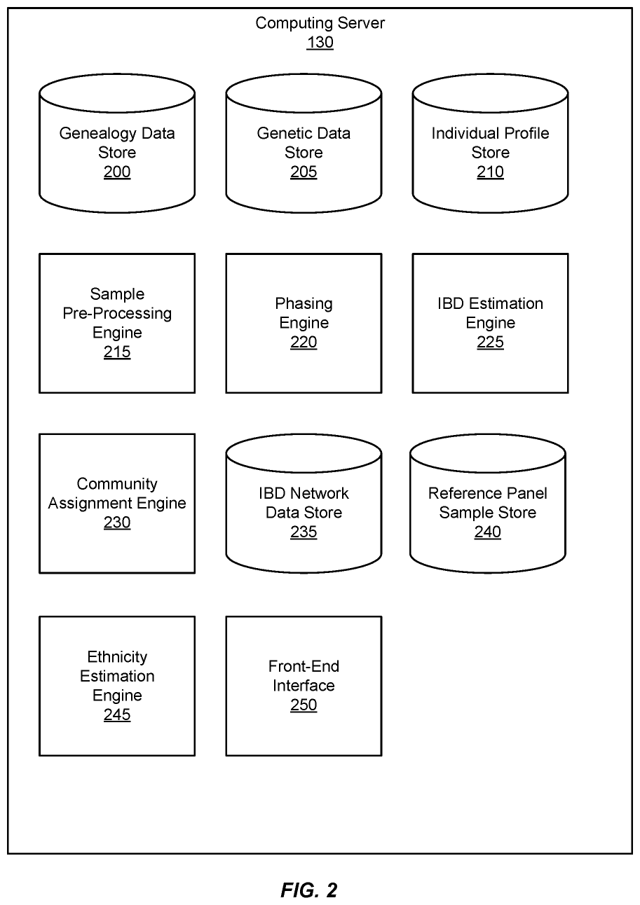

FIG. 2 is a block diagram of an architecture of an example computing server 130, in accordance with some embodiments. In the embodiment shown in FIG. 2, the computing server 130 includes a genealogy data store 200, a genetic data store 205, an individual profile store 210, a sample pre-processing engine 215, a phasing engine 220, an identity by descent (IBD) estimation engine 225, a community assignment engine 230, an IBD network data store 235, a reference panel sample store 240, an ethnicity estimation engine 245, and a front-end interface 250. The functions of the computing server 130 may be distributed among the elements in a different manner than described. In various embodiments, the computing server 130 may include different components and fewer or additional components. Each of the various data stores may be a single storage device, a server controlling multiple storage devices, or a distributed network that is accessible through multiple nodes (e.g., a cloud storage system).

The computing server 130 stores various data of different individuals, including genetic data, genealogy data, and survey response data. The computing server 130 processes the genetic data of users to identify shared identity-by-descent (IBD) segments between individuals. The genealogy data and survey response data may be part of user profile data. The amount and type of user profile data stored for each user may vary based on the information of a user, which is provided by the user as she creates an account and profile at a system operated by the computing server 130 and continues to build her profile, family tree, and social network at the system and to link her profile with her genetic data. Users may provide data via the user interface 115 of a client device 110. Initially and as a user continues to build her genealogical profile, the user may be prompted to answer questions related to the basic information of the user (e.g., name, date of birth, birthplace, etc.) and later on more advanced questions that may be useful for obtaining additional genealogy data. The computing server 130 may also include survey questions regarding various traits of the users such as the users' phenotypes, characteristics, preferences, habits, lifestyle, environment, etc.

Genealogy data may be stored in the genealogy data store 200 and may include various types of data that are related to tracing family relatives of users. Examples of genealogy data include names (first, last, middle, suffixes), gender, birth locations, date of birth, date of death, marriage information, spouse's information kinships, family history, dates and places for life events (e.g., birth and death), other vital data, and the like. In some instances, family history can take the form of a pedigree of an individual (e.g., the recorded relationships in the family). The family tree information associated with an individual may include one or more specified nodes. Each node in the family tree represents the individual, an ancestor of the individual who might have passed down genetic material to the individual, and the individual's other relatives including siblings, cousins, and offspring in some cases. Genealogy data may also include connections and relationships among users of the computing server 130. The information related to the connections among a user and her relatives that may be associated with a family tree may also be referred to as pedigree data or family tree data.

In addition to user-input data, genealogy data may also take other forms that are obtained from various sources such as public records and third-party data collectors. For example, genealogical records from public sources include birth records, marriage records, death records, census records, court records, probate records, adoption records, obituary records, etc. Likewise, genealogy data may include data from one or more family trees of an individual, the Ancestry World Tree system, a Social Security Death Index database, the World Family Tree system, a birth certificate database, a death certificate database, a marriage certificate database, an adoption database, a draft registration database, a veterans database, a military database, a property records database, a census database, a voter registration database, a phone database, an address database, a newspaper database, an immigration database, a family history records database, a local history records database, a business registration database, a motor vehicle database, and the like.

Furthermore, the genealogy data store 200 may also include relationship information inferred from the genetic data stored in the genetic data store 205 and information received from the individuals. For example, the relationship information may indicate which individuals are genetically related, how they are related, how many generations back they share common ancestors, lengths and locations of IBD segments shared, which genetic communities an individual is a part of, variants carried by the individual, and the like.

The computing server 130 maintains genetic datasets of individuals in the genetic data store 205. A genetic dataset of an individual may be a digital dataset of nucleotide data (e.g., SNP data) and corresponding metadata. A genetic dataset may contain data on the whole or portions of an individual's genome. The genetic data store 205 may store a pointer to a location associated with the genealogy data store 200 associated with the individual. A genetic dataset may take different forms. In some embodiments, a genetic dataset may take the form of a base pair sequence of the sequencing result of an individual. A base pair sequence dataset may include the whole genome of the individual (e.g., obtained from a whole-genome sequencing) or some parts of the genome (e.g., genetic loci of interest).

In another embodiment, a genetic dataset may take the form of sequences of genetic markers. Examples of genetic markers may include target SNP loci (e.g., allele sites) filtered from the sequencing results. A SNP locus that is single base pair long may also be referred to a SNP site. A SNP locus may be associated with a unique identifier. The genetic dataset may be in a form of diploid data that includes a sequencing of genotypes, such as genotypes at the target SNP loci, or the whole base pair sequence that includes genotypes at known SNP loci and other base pair sites that are not commonly associated with known SNPs. The diploid dataset may be referred to as a genotype dataset or a genotype sequence. Genotype may have a different meaning in various contexts. In one context, an individual's genotype may refer to a collection of diploid alleles of an individual. In other contexts, a genotype may be a pair of alleles present on two chromosomes for an individual at a given genetic marker such as a SNP site.

Genotype data for a SNP site may include a pair of alleles. The pair of alleles may be homozygous (e.g., A-A or G-G) or heterozygous (e.g., A-T, C-T). Instead of storing the actual nucleotides, the genetic data store 205 may store genetic data that are converted to bits. For a given SNP site, oftentimes only two nucleotide alleles (instead of all 4) are observed. As such, a 2-bit number may represent a SNP site. For example, 00 may represent homozygous first alleles, 11 may represent homozygous second alleles, and 01 or 10 may represent heterozygous alleles. A separate library may store what nucleotide corresponds to the first allele and what nucleotide corresponds to the second allele at a given SNP site.

A diploid dataset may also be phased into two sets of haploid data, one corresponding to a first parent side and another corresponding to a second parent side. The phased datasets may be referred to as haplotype datasets or haplotype sequences. Similar to genotype, haplotype may have a different meaning in various contexts. In one context, a haplotype may also refer to a collection of alleles that corresponds to a genetic segment. In other contexts, a haplotype may refer to a specific allele at a SNP site. For example, a sequence of haplotypes may refer to a sequence of alleles of an individual that are inherited from a parent.

The individual profile store 210 stores profiles and related metadata associated with various individuals appeared in the computing server 130. A computing server 130 may use unique individual identifiers to identify various users and other non-users that might appear in other data sources such as ancestors or historical persons who appear in any family tree or genealogy database. A unique individual identifier may be a hash of certain identification information of an individual, such as a user's account name, user's name, date of birth, location of birth, or any suitable combination of the information. The profile data related to an individual may be stored as metadata associated with an individual's profile. For example, the unique individual identifier and the metadata may be stored as a key-value pair using the unique individual identifier as a key.

An individual's profile data may include various kinds of information related to the individual. The metadata about the individual may include one or more pointers associating genetic datasets such as genotype and phased haplotype data of the individual that are saved in the genetic data store 205. The metadata about the individual may also be individual information related to family trees and pedigree datasets that include the individual. The profile data may further include declarative information about the user that was authorized by the user to be shared and may also include information inferred by the computing server 130. Other examples of information stored in a user profile may include biographic, demographic, and other types of descriptive information such as work experience, educational history, gender, hobbies, or preferences, location and the like. In some embodiments, the user profile data may also include one or more photos of the users and photos of relatives (e.g., ancestors) of the users that are uploaded by the users. A user may authorize the computing server 130 to analyze one or more photos to extract information, such as the user's or relative's appearance traits (e.g., blue eyes, curved hair, etc.), from the photos. The appearance traits and other information extracted from the photos may also be saved in the profile store. In some cases, the computing server may allow users to upload many different photos of the users, their relatives, and even friends. User profile data may also be obtained from other suitable sources, including historical records (e.g., records related to an ancestor), medical records, military records, photographs, other records indicating one or more traits, and other suitable recorded data.

For example, the computing server 130 may present various survey questions to its users from time to time. The responses to the survey questions may be stored at individual profile store 210. The survey questions may be related to various aspects of the users and the users' families. Some survey questions may be related to users' phenotypes, while other questions may be related to environmental factors of the users.

Survey questions may concern health or disease-related phenotypes, such as questions related to the presence or absence of genetic diseases or disorders, inheritable diseases or disorders, or other common diseases or disorders that have a family history as one of the risk factors, questions regarding any diagnosis of increased risk of any diseases or disorders, and questions concerning wellness-related issues such as a family history of obesity, family history of causes of death, etc. The diseases identified by the survey questions may be related to single-gene diseases or disorders that are caused by a single-nucleotide variant, an insertion, or a deletion. The diseases identified by the survey questions may also be multifactorial inheritance disorders that may be caused by a combination of environmental factors and genes. Examples of multifactorial inheritance disorders may include heart disease, Alzheimer's disease, diabetes, cancer, and obesity. The computing server 130 may obtain data on a user's disease-related phenotypes from survey questions about the health history of the user and her family and from health records uploaded by the user.

Survey questions also may be related to other types of phenotypes such as appearance traits of the users. A survey regarding appearance traits and characteristics may include questions related to eye color, iris pattern, freckles, chin types, finger length, dimple chin, earlobe types, hair color, hair curl, skin pigmentation, susceptibility to skin burn, bitter taste, male baldness, baldness pattern, presence of unibrow, presence of wisdom teeth, height, and weight. A survey regarding other traits also may include questions related to users' taste and smell such as the ability to taste bitterness, asparagus smell, cilantro aversion, etc. A survey regarding traits may further include questions related to users' body conditions such as lactose tolerance, caffeine consumption, malaria resistance, norovirus resistance, muscle performance, alcohol flush, etc. Other survey questions regarding a person's physiological or psychological traits may include vitamin traits and sensory traits such as the ability to sense an asparagus metabolite. Traits may also be collected from historical records, electronic health records and electronic medical records.

The computing server 130 also may present various survey questions related to the environmental factors of users. In this context, an environmental factor may be a factor that is not directly connected to the genetics of the users. Environmental factors may include users' preferences, habits, and lifestyles. For example, a survey regarding users' preferences may include questions related to things and activities that users like or dislike, such as types of music a user enjoys, dancing preference, party-going preference, certain sports that a user plays, video game preferences, etc. Other questions may be related to the users' diet preferences such as like or dislike a certain type of food (e.g., ice cream, egg). A survey related to habits and lifestyle may include questions regarding smoking habits, alcohol consumption and frequency, daily exercise duration, sleeping habits (e.g., morning person versus night person), sleeping cycles and problems, hobbies, and travel preferences. Additional environmental factors may include diet amount (calories, macronutrients), physical fitness abilities (e.g. stretching, flexibility, heart rate recovery), family type (adopted family or not, has siblings or not, lived with extended family during childhood), property and item ownership (has home or rents, has a smartphone or doesn't, has a car or doesn't).

Surveys also may be related to other environmental factors such as geographical, social-economic, or cultural factors. Geographical questions may include questions related to the birth location, family migration history, town, or city of users' current or past residence. Social-economic questions may be related to users' education level, income, occupations, self-identified demographic groups, etc. Questions related to culture may concern users' native language, language spoken at home, customs, dietary practices, etc. Other questions related to users' cultural and behavioral questions are also possible.

For any survey questions asked, the computing server 130 may also ask an individual the same or similar questions regarding the traits and environmental factors of the ancestors, family members, other relatives or friends of the individual. For example, a user may be asked about the native language of the user and the native languages of the user's parents and grandparents. A user may also be asked about the health history of his or her family members.

In addition to storing the survey data in the individual profile store 210, the computing server 130 may store some responses that correspond to data related to genealogical and genetics respectively to genealogy data store 200 and genetic data store 205.

The user profile data, photos of users, survey response data, the genetic data, and the genealogy data may be subject to the privacy and authorization setting of the users to specify any data related to the users that can be accessed, stored, obtained, or otherwise used. For example, when presented with a survey question, a user may select to answer or skip the question. The computing server 130 may present users from time to time information regarding users' selection of the extent of information and data shared. The computing server 130 also may maintain and enforce one or more privacy settings for users in connection with the access of the user profile data, photos, genetic data, and other sensitive data. For example, the user may pre-authorize the access to the data and may change the setting as wished. The privacy settings also may allow a user to specify (e.g., by opting out, by not opting in) whether the computing server 130 may receive, collect, log, or store particular data associated with the user for any purpose. A user may restrict her data at various levels. For example, on one level, the data may not be accessed by the computing server 130 for purposes other than displaying the data in the user's own profile. On another level, the user may authorize anonymization of her data and participate in studies and researches conducted by the computing server 130 such as a large-scale genetic study. On yet another level, the user may turn some portions of her genealogy data public to allow the user to be discovered by other users (e.g., potential relatives) and be connected to one or more family trees. Access or sharing of any information or data in the computing server 130 may also be subject to one or more similar privacy policies. A user's data and content objects in the computing server 130 may also be associated with different levels of restriction. The computing server 130 may also provide various notification features to inform and remind users of their privacy and access settings. For example, when privacy settings for a data entry allow a particular user or other entities to access the data, the data may be described as being “visible,” “public,” or other suitable labels, contrary to a “private” label.

In some cases, the computing server 130 may have a heightened privacy protection on certain types of data and data related to certain vulnerable groups. In some cases, the heightened privacy settings may strictly prohibit the use, analysis, and sharing of data related to a certain vulnerable group. In other cases, the heightened privacy settings may specify that data subject to those settings require prior approval for access, publication, or other use. In some cases, the computing server 130 may provide the heightened privacy as a default setting for certain types of data, such as genetic data or any data that the user marks as sensitive. The user may opt in to sharing of those data or change the default privacy settings. In other cases, the heightened privacy settings may apply across the board for all data of certain groups of users. For example, if computing server 130 determines that the user is a minor or has recognized that a picture of a minor is uploaded, the computing server 130 may designate all profile data associated with the minor as sensitive. In those cases, the computing server 130 may have one or more extra steps in seeking and confirming any sharing or use of the sensitive data.

The sample pre-processing engine 215 receives and pre-processes data received from various sources to change the data into a format used by the computing server 130. For genealogy data, the sample pre-processing engine 215 may receive data from an individual via the user interface 115 of the client device 110. To collect the user data (e.g., genealogical and survey data), the computing server 130 may cause an interactive user interface on the client device 110 to display interface elements in which users can provide genealogy data and survey data. Additional data may be obtained from scans of public records. The data may be manually provided or automatically extracted via, for example, optical character recognition (OCR) performed on census records, town or government records, or any other item of printed or online material. Some records may be obtained by digitalizing written records such as older census records, birth certificates, death certificates, etc.

The sample pre-processing engine 215 may also receive raw data from genetic data extraction service server 125. The genetic data extraction service server 125 may perform laboratory analysis of biological samples of users and generate sequencing results in the form of digital data. The sample pre-processing engine 215 may receive the raw genetic datasets from the genetic data extraction service server 125. Most of the mutations that are passed down to descendants are related to single-nucleotide polymorphism (SNP). SNP is a substitution of a single nucleotide that occurs at a specific position in the genome. The sample pre-processing engine 215 may convert the raw base pair sequence into a sequence of genotypes of target SNP sites. Alternatively, the pre-processing of this conversion may be performed by the genetic data extraction service server 125. The sample pre-processing engine 215 identifies autosomal SNPs in an individual's genetic dataset. In some embodiments, the SNPs may be autosomal SNPs. In some embodiments, 700,000 SNPs may be identified in an individual's data and may be stored in genetic data store 205. Alternatively, in some embodiments, a genetic dataset may include at least 10,000 SNP sites. In another embodiment, a genetic dataset may include at least 100,000 SNP sites. In yet another embodiment, a genetic dataset may include at least 300,000 SNP sites. In yet another embodiment, a genetic dataset may include at least 1,000,000 SNP sites. The sample pre-processing engine 215 may also convert the nucleotides into bits. The identified SNPs, in bits or in other suitable formats, may be provided to the phasing engine 220 which phases the individual's diploid genotypes to generate a pair of haplotypes for each user.

The phasing engine 220 phases diploid genetic dataset into a pair of haploid genetic datasets and may perform imputation of SNP values at certain sites whose alleles are missing. An individual's haplotype may refer to a collection of alleles (e.g., a sequence of alleles) that are inherited from a parent.

Phasing may include a process of determining the assignment of alleles (particularly heterozygous alleles) to chromosomes. Owing to sequencing conditions and other constraints, a sequencing result often includes data regarding a pair of alleles at a given SNP locus of a pair of chromosomes but may not be able to distinguish which allele belongs to which specific chromosome. The phasing engine 220 uses a genotype phasing algorithm to assign one allele to a first chromosome and another allele to another chromosome. The genotype phasing algorithm may be developed based on an assumption of linkage disequilibrium (LD), which states that haplotype in the form of a sequence of alleles tends to cluster together. The phasing engine 220 is configured to generate phased sequences that are also commonly observed in many other samples. Put differently, haplotype sequences of different individuals tend to cluster together. A haplotype-cluster model may be generated to determine the probability distribution of a haplotype that includes a sequence of alleles. The haplotype-cluster model may be trained based on labeled data that includes known phased haplotypes from a trio (parents and a child). A trio is used as a training sample because the correct phasing of the child is almost certain by comparing the child's genotypes to the parent's genetic datasets. The haplotype-cluster model may be generated iteratively along with the phasing process with a large number of unphased genotype datasets. The haplotype-cluster model may also be used to impute one or more missing data.

By way of example, the phasing engine 220 may use a directed acyclic graph model such as a hidden Markov model (HMM) to perform the phasing of a target genotype dataset. The directed acyclic graph may include multiple levels, each level having multiple nodes representing different possibilities of haplotype clusters. An emission probability of a node, which may represent the probability of having a particular haplotype cluster given an observation of the genotypes may be determined based on the probability distribution of the haplotype-cluster model. A transition probability from one node to another may be initially assigned to a non-zero value and be adjusted as the directed acyclic graph model and the haplotype-cluster model are trained. Various paths are possible in traversing different levels of the directed acyclic graph model. The phasing engine 220 determines a statistically likely path, such as the most probable path or a probable path that is at least more likely than 95% of other possible paths, based on the transition probabilities and the emission probabilities. A suitable dynamic programming algorithm such as the Viterbi algorithm may be used to determine the path. The determined path may represent the phasing result. U.S. Pat. No. 10,679,729, entitled “Haplotype Phasing Models,” granted on Jun. 9, 2020, describes example embodiments of haplotype phasing. Other example phasing embodiments are described in U.S. Patent Application Publication No. US 2021/0034647, entitled “Clustering of Matched Segments to Determine Linkage of Dataset in a Database,” published on Feb. 4, 2021.

The IBD estimation engine 225 estimates the amount of shared genetic segments between a pair of individuals based on phased genotype data (e.g., haplotype datasets) that are stored in the genetic data store 205. IBD segments may be segments identified in a pair of individuals that are putatively determined to be inherited from a common ancestor. The IBD estimation engine 225 retrieves a pair of haplotype datasets for each individual. The IBD estimation engine 225 may divide each haplotype dataset sequence into a plurality of windows. Each window may include a fixed number of SNP sites (e.g., about 100 SNP sites). The IBD estimation engine 225 identifies one or more seed windows in which the alleles at all SNP sites in at least one of the phased haplotypes between two individuals are identical. The IBD estimation engine 225 may expand the match from the seed windows to nearby windows until the matched windows reach the end of a chromosome or until a homozygous mismatch is found, which indicates the mismatch is not attributable to potential errors in phasing or imputation. The IBD estimation engine 225 determines the total length of matched segments, which may also be referred to as IBD segments. The length may be measured in the genetic distance in the unit of centimorgans (cM). A unit of centimorgan may be a genetic length. For example, two genomic positions that are one cM apart may have a 1% chance during each meiosis of experiencing a recombination event between the two positions. The computing server 130 may save data regarding individual pairs who share a length of IBD segments exceeding a predetermined threshold (e.g., 6 cM), in a suitable data store such as in the genealogy data store 200. U.S. Pat. No. 10,114,922, entitled “Identifying Ancestral Relationships Using a Continuous stream of Input,” granted on Oct. 30, 2018, and U.S. Pat. No. 10,720,229, entitled “Reducing Error in Predicted Genetic Relationships,” granted on Jul. 21, 2020, describe example embodiments of IBD estimation.

The IBD estimation engine 225 may also use an encoding algorithm to process the genetic data of the individuals. The encoding algorithm may serve as a formal scan or a pre-scan before another IBD determination algorithm is applied. The encoding algorithm encodes a genotype dataset into a pair of bitmaps. Each bitmap encodes locations of homozygous alleles in the genotype so that locations of homozygous mismatch may be quickly identified when comparing the bitmaps of two individuals. U.S. patent application Ser. No. 17/825,220, entitled “Identification of Matched Segmented in Paired Datasets,” filed on May 26, 2022, describe some example encoding algorithms and is incorporated by reference for all purposes.

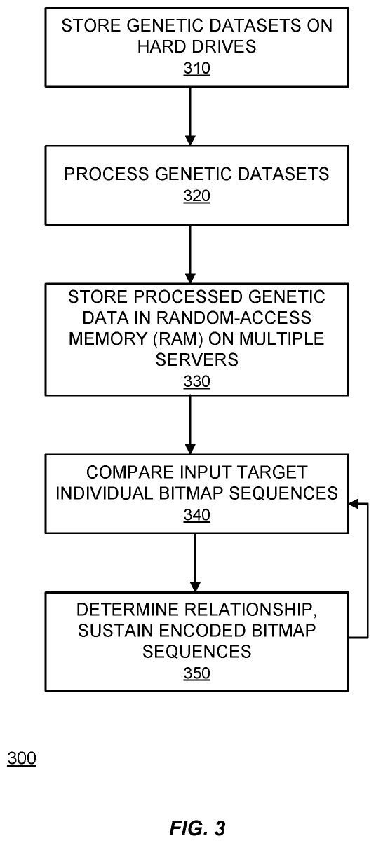

The IBD estimation engine 225 may be a sub-server of the computing server and may take the form of a distributed system with a plurality of servers working in parallel to speed up the identification of genetic matches of a target individual. Examples of structure and process for determining IBD segments and matches using a plurality of servers are discussed in further detail in FIG. 3 through FIG. 7B.

Typically, individuals who are closely related share a relatively large number of IBD segments, and the IBD segments tend to have longer lengths (individually or in aggregate across one or more chromosomes). In contrast, individuals who are more distantly related share relatively fewer IBD segments, and these segments tend to be shorter (individually or in aggregate across one or more chromosomes). For example, while close family members often share upwards of 71 cM of IBD (e.g., third cousins), more distantly related individuals may share less than 12 cM of IBD. The extent of relatedness in terms of IBD segments between two individuals may be referred to as IBD affinity. For example, the IBD affinity may be measured in terms of the length of IBD segments shared between two individuals.

Community assignment engine 230 assigns individuals to one or more genetic communities based on the genetic data of the individuals. A genetic community may correspond to an ethnic origin or a group of people descended from a common ancestor. The granularity of genetic community classification may vary depending on embodiments and methods used to assign communities. For example, in some embodiments, the communities may be African, Asian, European, etc. In another embodiment, the European community may be divided into Irish, German, Swedes, etc. In yet another embodiment, the Irish may be further divided into Irish in Ireland, Irish immigrated to America in 1800, Irish immigrated to America in 1900, etc. The community classification may also depend on whether a population is admixed or unadmixed. For an admixed population, the classification may further be divided based on different ethnic origins in a geographical region.

Community assignment engine 230 may assign individuals to one or more genetic communities based on their genetic datasets using machine learning models trained by unsupervised learning or supervised learning. In an unsupervised approach, the community assignment engine 230 may generate data representing a partially connected undirected graph. In this approach, the community assignment engine 230 represents individuals as nodes. Some nodes are connected by edges whose weights are based on IBD affinity between two individuals represented by the nodes. For example, if the total length of two individuals' shared IBD segments does not exceed a predetermined threshold, the nodes are not connected. The edges connecting two nodes are associated with weights that are measured based on the IBD affinities. The undirected graph may be referred to as an IBD network. The community assignment engine 230 uses clustering techniques such as modularity measurement (e.g., the Louvain method) to classify nodes into different clusters in the IBD network. Each cluster may represent a community. The community assignment engine 230 may also determine sub-clusters, which represent sub-communities. The computing server 130 saves the data representing the IBD network and clusters in the IBD network data store 235. U.S. Pat. No. 10,223,498, entitled “Discovering Population Structure from Patterns of Identity-By-Descent,” granted on Mar. 5, 2019, describes example embodiments of community detection and assignment.

The community assignment engine 230 may also assign communities using supervised techniques. For example, genetic datasets of known genetic communities (e.g., individuals with confirmed ethnic origins) may be used as training sets that have labels of the genetic communities. Supervised machine learning classifiers, such as logistic regressions, support vector machines, random forest classifiers, and neural networks may be trained using the training set with labels. A trained classifier may distinguish binary or multiple classes. For example, a binary classifier may be trained for each community of interest to determine whether a target individual's genetic dataset belongs or does not belong to the community of interest. A multi-class classifier such as a neural network may also be trained to determine whether the target individual's genetic dataset most likely belongs to one of several possible genetic communities.

Reference panel sample store 240 stores reference panel samples for different genetic communities. A reference panel sample is a genetic data of an individual whose genetic data is the most representative of a genetic community. The genetic data of individuals with the typical alleles of a genetic community may serve as reference panel samples. For example, some alleles of genes may be over-represented (e.g., being highly common) in a genetic community. Some genetic datasets include alleles that are commonly present among members of the community. Reference panel samples may be used to train various machine learning models in classifying whether a target genetic dataset belongs to a community, determining the ethnic composition of an individual, and determining the accuracy of any genetic data analysis, such as by computing a posterior probability of a classification result from a classifier.

A reference panel sample may be identified in different ways. In some embodiments, an unsupervised approach in community detection may apply the clustering algorithm recursively for each identified cluster until the sub-clusters contain a number of nodes that are smaller than a threshold (e.g., contains fewer than 1000 nodes). For example, the community assignment engine 230 may construct a full IBD network that includes a set of individuals represented by nodes and generate communities using clustering techniques. The community assignment engine 230 may randomly sample a subset of nodes to generate a sampled IBD network. The community assignment engine 230 may recursively apply clustering techniques to generate communities in the sampled IBD network. The sampling and clustering may be repeated for different randomly generated sampled IBD networks for various runs. Nodes that are consistently assigned to the same genetic community when sampled in various runs may be classified as a reference panel sample. The community assignment engine 230 may measure the consistency in terms of a predetermined threshold. For example, if a node is classified to the same community 95% (or another suitable threshold) of the times whenever the node is sampled, the genetic dataset corresponding to the individual represented by the node may be regarded as a reference panel sample. Additionally, or alternatively, the community assignment engine 230 may select N most consistently assigned nodes as a reference panel for the community.

Other ways to generate reference panel samples are also possible. For example, the computing server 130 may collect a set of samples and gradually filter and refine the samples until high-quality reference panel samples are selected. For example, a candidate reference panel sample may be selected from an individual whose recent ancestors are born at a certain birthplace. The computing server 130 may also draw sequence data from the Human Genome Diversity Project (HGDP). Various candidates may be manually screened based on their family trees, relatives' birth location, and other quality control. Principal component analysis may be used to create clusters of genetic data of the candidates. Each cluster may represent an ethnicity. The predictions of the ethnicity of those candidates may be compared to the ethnicity information provided by the candidates to perform further screening.

The ethnicity estimation engine 245 estimates the ethnicity composition of a genetic dataset of a target individual. The genetic datasets used by the ethnicity estimation engine 245 may be genotype datasets or haplotype datasets. For example, the ethnicity estimation engine 245 estimates the ancestral origins (e.g., ethnicity) based on the individual's genotypes or haplotypes at the SNP sites. To take a simple example of three ancestral populations corresponding to African, European and Native American, an admixed user may have nonzero estimated ethnicity proportions for all three ancestral populations, with an estimate such as [0.05, 0.65, 0.30], indicating that the user's genome is 5% attributable to African ancestry, 65% attributable to European ancestry and 30% attributable to Native American ancestry. The ethnicity estimation engine 245 generates the ethnic composition estimate and stores the estimated ethnicities in a data store of computing server 130 with a pointer in association with a particular user.

In some embodiments, the ethnicity estimation engine 245 divides a target genetic dataset into a plurality of windows (e.g., about 1000 windows). Each window includes a small number of SNPs (e.g., 300 SNPs). The ethnicity estimation engine 245 may use a directed acyclic graph model to determine the ethnic composition of the target genetic dataset. The directed acyclic graph may represent a trellis of an inter-window hidden Markov model (HMM). The graph includes a sequence of a plurality of node groups. Each node group, representing a window, includes a plurality of nodes. The nodes represent different possibilities of labels of genetic communities (e.g., ethnicities) for the window. A node may be labeled with one or more ethnic labels. For example, a level includes a first node with a first label representing the likelihood that the window of SNP sites belongs to a first ethnicity and a second node with a second label representing the likelihood that the window of SNPs belongs to a second ethnicity. Each level includes multiple nodes so that there are many possible paths to traverse the directed acyclic graph.

The nodes and edges in the directed acyclic graph may be associated with different emission probabilities and transition probabilities. An emission probability associated with a node represents the likelihood that the window belongs to the ethnicity labeling the node given the observation of SNPs in the window. The ethnicity estimation engine 245 determines the emission probabilities by comparing SNPs in the window corresponding to the target genetic dataset to corresponding SNPs in the windows in various reference panel samples of different genetic communities stored in the reference panel sample store 240. The transition probability between two nodes represents the likelihood of transition from one node to another across two levels. The ethnicity estimation engine 245 determines a statistically likely path, such as the most probable path or a probable path that is at least more likely than 95% of other possible paths, based on the transition probabilities and the emission probabilities. A suitable dynamic programming algorithm such as the Viterbi algorithm or the forward-backward algorithm may be used to determine the path. After the path is determined, the ethnicity estimation engine 245 determines the ethnic composition of the target genetic dataset by determining the label compositions of the nodes that are included in the determined path. U.S. Pat. No. 10,558,930, entitled “Local Genetic Ethnicity Determination System,” granted on Feb. 11, 2020 and U.S. Pat. No. 10,692,587, granted on Jun. 23, 2020, entitled “Global Ancestry Determination System” describe different example embodiments of ethnicity estimation.

The front-end interface 250 displays various results determined by the computing server 130. The results and data may include the IBD affinity between a user and another individual, the community assignment of the user, the ethnicity estimation of the user, phenotype prediction and evaluation, genealogy data search, family tree and pedigree, relative profile and other information. The front-end interface 250 may allow users to manage their profile and data trees (e.g., family trees). The users may view various public family trees stored in the computing server 130 and search for individuals and their genealogy data via the front-end interface 250. The computing server 130 may suggest or allow the user to manually review and select potentially related individuals (e.g., relatives, ancestors, close family members) to add to the user's data tree. The front-end interface 250 may be a graphical user interface (GUI) that displays various information and graphical elements. The front-end interface 250 may take different forms. In one case, the front-end interface 250 may be a software application that can be displayed on an electronic device such as a computer or a smartphone. The software application may be developed by the entity controlling the computing server 130 and be downloaded and installed on the client device 110. In another case, the front-end interface 250 may take the form of a webpage interface of the computing server 130 that allows users to access their family tree and genetic analysis results through web browsers. In yet another case, the front-end interface 250 may provide an application program interface (API).

The tree management engine 260 performs computations and other processes related to users' management of their data trees such as family trees. The tree management engine 260 may allow a user to build a data tree from scratch or to link the user to existing data trees. In some embodiments, the tree management engine 260 may suggest a connection between a target individual and a family tree that exists in the family tree database by identifying potential family trees for the target individual and identifying one or more most probable positions in a potential family tree. A user (target individual) may wish to identify family trees to which he or she may potentially belong. Linking a user to a family tree or building a family may be performed automatically, manually, or using techniques with a combination of both. In an embodiment of an automatic tree matching, the tree management engine 260 may receive a genetic dataset from the target individual as input and search related individuals that are IBD-related to the target individual. The tree management engine 260 may identify common ancestors. Each common ancestor may be common to the target individual and one of the related individuals. The tree management engine 260 may in turn output potential family trees to which the target individual may belong by retrieving family trees that include a common ancestor and an individual who is IBD-related to the target individual. The tree management engine 260 may further identify one or more probable positions in one of the potential family trees based on information associated with matched genetic data between the target individual and those in the potential family trees through one or more machine learning models or other heuristic algorithms. For example, the tree management engine 260 may try putting the target individual in various possible locations in the family tree and determine the highest probability position(s) based on the genetic dataset of the target individual and genetic datasets available for others in the family tree and based on genealogy data available to the tree management engine 260. The tree management engine 260 may provide one or more family trees from which the target individual may select. For a suggested family tree, the tree management engine 260 may also provide information on how the target individual is related to other individuals in the tree. In a manual tree building, a user may browse through public family trees and public individual entries in the genealogy data store 200 and individual profile store 210 to look for potential relatives that can be added to the user's family tree. The tree management engine 260 may automatically search, rank, and suggest individuals for the user conduct manual reviews as the user makes progress in the front-end interface 250 in building the family tree.

As used herein, “pedigree” and “family tree” may be interchangeable and may refer to a family tree chart or pedigree chart that shows, diagrammatically, family information, such as family history information, including parentage, offspring, spouses, siblings, or otherwise for any suitable number of generations and/or people, and/or data pertaining to persons represented in the chart. U.S. Patent Publication Application No., entitled “Linking Individual Datasets to a Database,” US2021/0216556, published on Jul. 15, 2021, describes example embodiments of how an individual may be linked to existing family trees.

### Example IBD Determination Process Using a Distributed System

Embodiments described herein are related to a distributed system that speeds up the process of identification matches of various target individuals based on shared IBD segments. The distributed system may include a plurality of servers working in parallel. The servers store processed genetic data in structured and read-to-compare forms in the random-access memory (RAM) of the servers and sustain those data in RAM for later comparison. The process of identifying IBD matched segments are sped up through a combination of data processing and better hardware allocation. Details of the hardware structure and software algorithm implementation the process are discussed in FIG. 3 through FIG. 7B.

FIG. 3 is a flowchart depicting an example process 300 for identifying a target individual's potential relatives based on the lengths of shared IBD segments, in accordance with some embodiments. The process may be performed by one or more engines of the computing server 130 illustrated in FIG. 2, such as the IBD estimation engine 225. The process 300 may be embodied as a software algorithm that may be stored as computer instructions that are executable by one or more processors. The instructions, when executed by the processors, cause the processors to perform various steps in the process 300. In various embodiments, the process may include additional, fewer, or different steps.

In some embodiments, the process 300 includes storing genetic datasets on hard drives (step 310). Step 310 may be performed using computing server 130; for example, genetic datasets may be stored on one or more storage media, such as hard drives, in genetic data store 205. The genetic datasets may include genetic data associated with various individual end users of the computing server 130, who may or may not have built family trees using the computing server 130. The genetic datasets may further include genetic data from predetermined reference panels of individuals with a known shared location, individuals with a known shared ethnicity, or individuals common to some other criterium. The reference panels may be those stored in reference panel sample store 240. In some embodiments, the genetic datasets of these individuals are stored on the hard drives of servers. In various embodiments, the genetic datasets may include a large amount of data. For example, the genetic datasets may include data of more than 10,000 individuals. In some embodiments, the genetic datasets may include data of more than 10,000 individuals. In some embodiments, the genetic datasets may include data of more than 100,000 individuals. In some embodiments, the genetic datasets may include data of more than 500,000 individuals. In some embodiments, the genetic datasets may include data of more than 1,000,000 individuals.

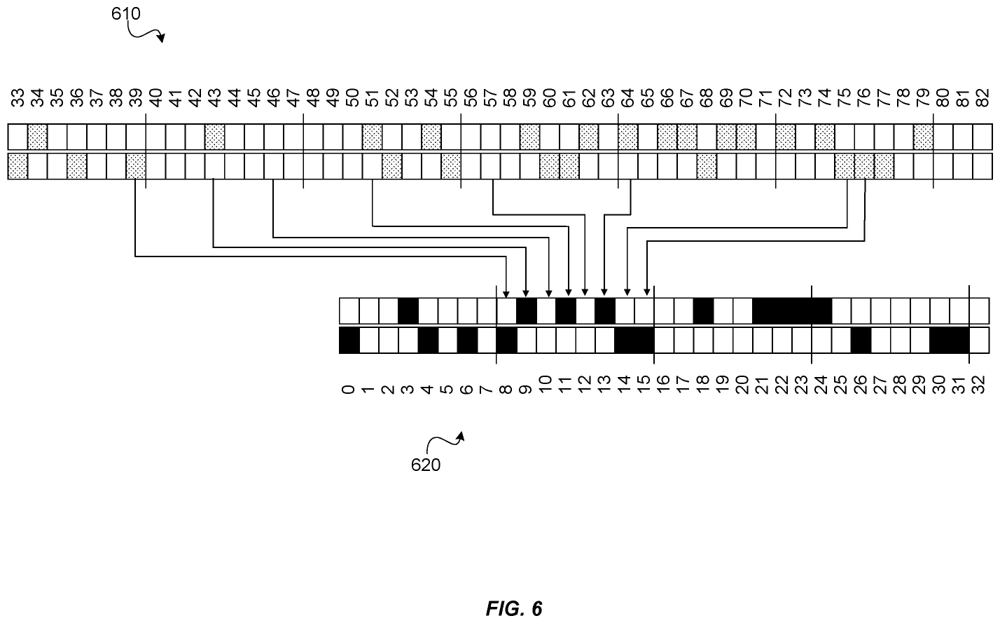

With continued reference to FIG. 3, the process 300 can additionally include processing the genetic datasets of individuals stored on the hard drives (step 320). How the genetic data are processed at step 320 may vary depending on the type of IBD determination algorithm used. For example, in some embodiments, the IBD algorithm may include GERMLINE or J-GERMLINE that is discussed in the IBD estimation engine 225. In some embodiments, the IBD algorithm may include an encoding algorithm described in U.S. patent application Ser. No. 17/825,220, entitled “Identification of Matched Segmented in Paired Datasets,” filed on May 26, 2022. As provided therein, an exemplary encoding scheme may involve generating bitmap pair sequences for each individual and can include defining encoding values based on homogeneity between a pair of data value sequences. An example of such encoding scheme is discussed and illustrated in further detail with respect to FIG. 6. Briefly, the pair of encoded target bitmap sequences includes a first encoded target bitmap sequence that encodes a first type of homogeneous locations and a second encoded target bitmap sequence that encodes a second type of homogeneous locations. This disclosed encoding scheme includes comparing the pair of encoded target bitmap sequences with other pairs of encoded bitmap sequences to identify homogeneous mismatched locations. The other encoded bitmap sequences may be generated from the other datasets in the database using the encoding scheme. A homogeneous mismatched location may be a location where the target dataset and the other dataset in comparison are both homogeneous but have different types of homogeneity at the location. This exemplary method may further identify a matched segment between the target dataset and one of the other datasets based on the homogeneous mismatched locations identified with the matched segment being contained within two homogeneous mismatched locations.

With continued reference to FIG. 3, the process 300 can additionally include storing processed data in random-access memory (RAM) on multiple servers (step 330). RAM is a volatile memory that has a faster access speed than hard drives, which are non-volatile. RAM may also be referred to cache in this context. At step 330, the processed data may be haplotypes in the cases of GERMLINE or J-GERMLINE; the processed data may also be encoded bitmap sequences in the case of using an encoding algorithm. In some embodiments, the bitmap sequences are bitmap pair sequences for individuals from a predetermined selected reference panel. By storing the encoded bitmap sequences in RAM, a computing system, such as the computing server 130, can avoid the process of encoding and loading genetic datasets every time they aim to determine relationships. In some embodiments, the servers each store encoded data for non-overlapping sets of individuals. The servers may store the encoded datasets for an indefinite amount of time, a user-defined amount of time, or a pre-set amount of time such as one hour, one day, one week, or another length of time. According to some embodiments, each server simultaneously sustains a portion of the processed data on their RAM. For example, the entirety of the processed data may correspond to data of M individuals (can be in thousands or even millions) and may be divided among N servers. Each server may store M/N portion of the processed data.

With continued reference to FIG. 3, the process 300 can additionally include comparing an input data of a target individual to processed data stored in the RAM of each server (step 340). In some instances, the processed data acted upon at step 340 corresponds to the genetic datasets of different individuals. It should be appreciated in embodiments where the encoding algorithm is used, the input target individual data utilized with step 340 may be encoded as bitmap sequences. In such instances, the computing system, such as server 130, can use the encoded bitmap sequences of individuals stored in the RAM of servers to match against the input target individual's bitmap sequences. The comparison process of step 340 may be performed by different servers in parallel. For example, a first server may compare the target individual data to the processed data that correspond to a first subset of individuals (e.g., first M/Nth individuals). A second server may compare the target individual data to the processed data that correspond to a second subset of individuals (e.g., second M/Nth individuals). Since each processed dataset is stored in the RAM of a server, the comparison is sped up compared to storing the processed dataset in a hard drive.

With continued reference to FIG. 3, the process 300 can additionally include determine the relationship between the target individual and the processed data (e.g., bitmap sequences) for multiple individuals stored in RAM (step 350). After the relationship determination is completed, the servers continue to sustain the encoded bitmap sequences for the multiple individuals stored in the RAM. When a subsequent target individual is input for comparison to the multiple individuals, the flowchart goes back to step 340 and the new target individual is compared against the bitmap sequences stored in the flash memories of multiple servers. The relationship for the new target individual is determined and the encoded bitmap sequences of the multiple individuals are still sustained, creating a loop between 340 and 350.

### Determining Relationships Based on Shared IBD Segments

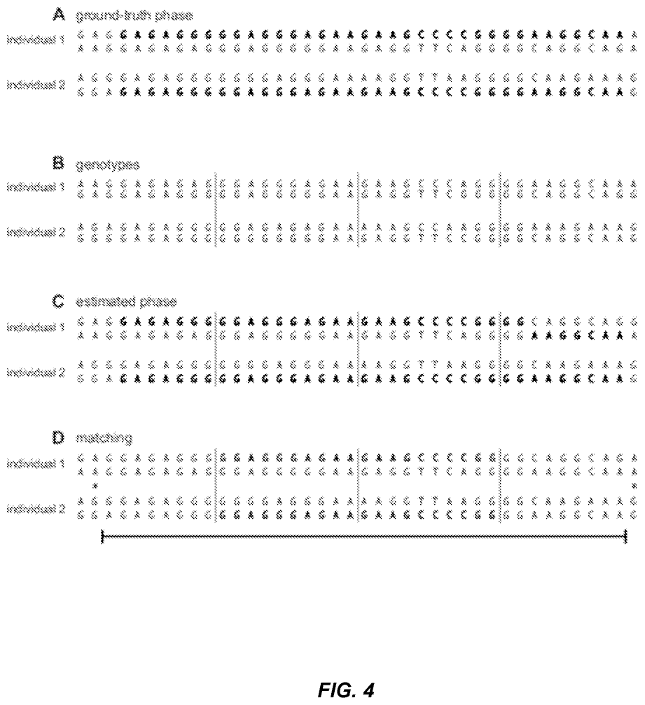

FIG. 4 is a conceptual diagram graphically illustrating a process of finding shared IBD segments between two individuals, in accordance with some embodiments. After the phasing is completed by the phasing engine 220, the computing server 130 has assigned the two allele copies of each genetic marker to each of an individual's two chromosomes. To determine relationships among individuals, the computing server 130 may identify identical DNA sequences between all pairs of individuals in the genetic data store 205. This is challenging because it involves comparing a very large number of sequences. For example, the computing server 130 may store more than 1.2 million genotyped DNA samples in genetic data store 205. This example number represents more than 700 billion pairs of individuals to check for matching segments. An additional complication is that the database may not be static—it may be continuously growing as additional users incorporate their genetic data into the genetic data store.

Quantitative geneticists have developed software such as GERMLINE (Gusev et al., 2009) and Parente (Rodriguez et al., 2015) to identify matches in a large number of genotype samples. However, even this type of software is slow to operate on a massive customer database of computing server 130. In some embodiment, the computing server 130 may use software similar to GERMLINE that allows the computing server 130 to quickly detect matches in hundreds of thousands of phased genotypes, as well as quickly identify matches as new customers enter the database each day.

By way of example, after the computing server 130 has estimated the phase of each genotype sample, the computing server 130 may turn to the problem of finding IBD segments, or “matches,” shared by pairs of samples. This effectively reduces to the problem of finding long sequences (strings of A's, T's, G's and C's) that are identical in pairs of chromosomes. However, there are several practical issues that arise due to the peculiarities of genetic data, as well as the size of our data set, that make this problem more complex than it might first appear.

In some embodiment, finding of IBD match may be divided into several steps. An example of the process is graphically illustrated with FIG. 4.

In the first step, the computing server 130 may subdivide each chromosome into short segments, which may be called “windows.” For example, windows may contain 96 SNPs. This number may be chosen to balance computational cost and accuracy.

In the second step, for each pair of individuals, the computing server 130 may identify windows in which the alleles at all SNPs in one of the individual's two phased haplotypes are identical to all the alleles at the same positions in one of the other individual's phased haplotypes. These may be referred to as “seed matches” (see FIG. 4, section D). In the second step depicted in step D, for each pair of individuals, the computing server 130 may identify windows in which the alleles at all SNPs in one of the individual's two phased haplotypes are identical to all the alleles at the same positions in one of the other individual's phased haplotypes. The highlighted portions of step D would be grouped together in the storage of genetic datasets. This makes it more efficient to pair up DNA segments for analysis without comparing all the DNA of individuals that do not share identical DNA with a query individual.

In the third step, for each seed match, the computing server 130 may attempt to extend the seed match in both directions along the chromosome until (a) the beginning or end of the chromosome is reached, or (b) a homozygous mismatch is detected. A homozygous mismatch is a pair of genotypes at the same SNP that are incompatible regardless of how they are phased (for example, AA and GG). The estimated IBD region is defined by the start and end positions of the SNPs included in the extended segment.

In the fourth step, the computing server 130 may calculate the length of the candidate matching segment in terms of genetic distance, measured in centimorgans (cM). Genetic distance is proportional to the expected rate of recombination along that stretch of chromosome. Since individual chromosomes accumulate recombination events through successive generations of inheritance, IBD segments spanning large genetic distances suggest more recent inheritance.

In the fifth step, if the segment is longer than a threshold (e.g., 6 cM), the computing server 130 may store that segment as a match in a database.

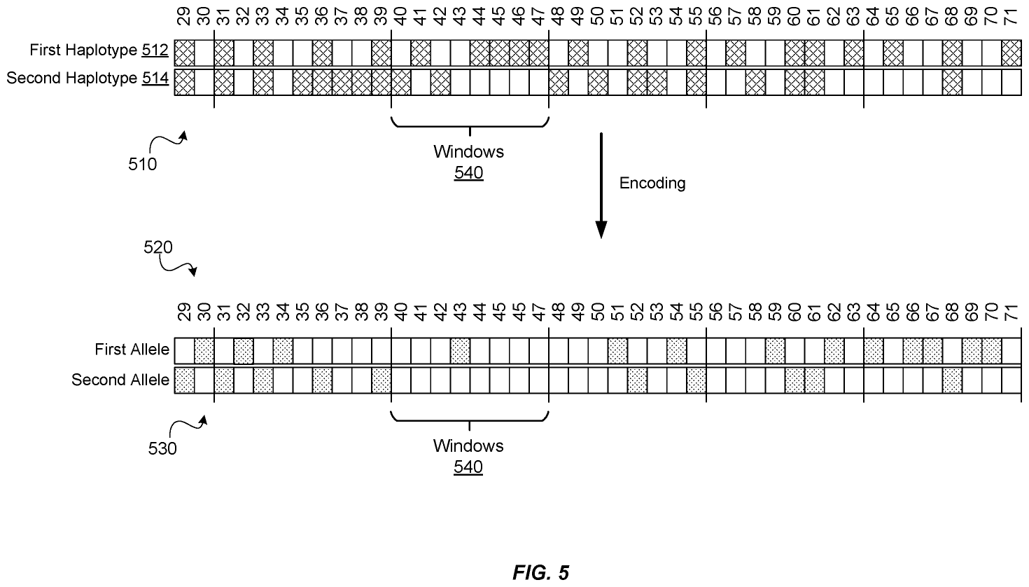

FIG. 5 shows an example of encoding genotype data that is used in an encoding algorithm approach in determining IBD, according to some embodiments. Example phased genotype data 512 and 514 are represented, although in various embodiments phasing may not be needed for the use of the encoding algorithm. At each position of sequence 512 or 514, a shaded block represents one allele and the white block represents the other allele. Each biallelic SNP is encoded arbitrarily (without a loss of generality, white blocks could represent the major allele and shaded blocks could represent the minor allele). For example, individual A with the genotype shown in FIG. 5 is homozygous for the major allele at position 34, heterozygous at position 35, and homozygous for the minor allele at position 36. Each position (SNP position) is numbered in order and lines are drawn between windows of 8 SNPs. Elements 520 and 530 are an example of a bitmap pair. For example, homozygous bitmap sequences 520 and 530 for an individual A are shown. The first homozygosity bitmap sequence 520 for individual A is encoded as a value that is graphically illustrated as shaded if individual A is homozygous for the major allele (e.g., the first allele value at the corresponding position. The second homozygous bitmap sequence 530 for individual A is shaded if individual A is homozygous for the minor allele (e.g., the second allele value) at the corresponding position.

The encoding for the window that includes position 48 through position 55 is discussed in further detail as an example. Position 48 is heterozygous. Hence, both bitmap sequences 520 and 530 are encoded with white blocks (e.g., second value, or 0) at position 48. The same encoding is performed for positions 49 and 50 as both positions are heterozygous. For position 51, it is a homozygous position with the major allele (first allele). Hence, the bitmap sequence 520 is encoded with a shaded block (e.g., first value, or 1) at position 51 but the bitmap sequence 530 is still encoded with a white block because, for the bitmap sequence 530, either a heterozygous position or a homozygous position with the major allele is encoded with a white block. For the bitmap sequence 530, only a homozygous position with the minor allele is encoded with a shaded block. For positions 60 and 61, both positions are homozygous with the minor alleles (second allele). Hence, the bitmap sequence 530 is encoded with shaded blocks at both positions 60 and 61. The bitmap sequence 520 is encoded with white blocks at positions 60 and 61 because either a heterozygous position or a homozygous position with the minor allele is encoded with a white block in bitmap 520. For position 62, it is a homozygous position with the major allele. Hence, the bitmap sequence 520 is encoded with a shaded block at position 62 and the bitmap sequence 530 is encoded with a white block at position 62. Position 63 is heterozygous. Hence, both bitmap sequences 520 and 530 are encoded with white blocks. As the encoding scheme is based on homozygosity, the genetic dataset 510 may be phased or not phased.

FIG. 6 is a conceptual diagram illustrating an example sampling process for turning a pair of bitmap sequences 610 into a pair of sparse bitmap sequences 620, in accordance with some embodiments. The computing server 130 may sample some of the locations of the pair of bitmap sequences 610 and generate a shorter pair of bitmap sequences that may be referred to as a pair of sparse bitmap sequences 620. The sparse bitmap sequences 620 are shorter than the bitmap sequences 610 because only some of the locations in the bitmap sequences 610 are sampled to the sparse bitmap sequences 620. FIG. 6 shows an example with less than 100 bases, but in practice the bitmap sequences 610 may be millions of bases long and the sparse bitmap sequences 620 may still be millions of bases long (despite being shorter than bitmap sequences 610) or hundreds of thousands of bases long. The numerical positions of the bitmap sequences 610 and the sparse bitmap sequences 620 do not need to correspond. The computing server 130 stores a mapping of the positions. For example, position 39 in the bitmap sequences 610 is sampled at position 8 in the sparse bitmap sequences 620. The sparse bitmap sequences 620 may also be divided in windows but the window lengths in the bitmap sequences 610 and in the sparse bitmap sequences 620 are necessarily related. In some embodiments, both types of sequences use the window length of 8 so that each window may carry a byte of data.

In sampling various positions, the encoded homozygosity values in the bitmap sequences 610 are carried to the sparse bitmap sequences 620. For example, positions 39, 43, 47, 51, 57, 64, 75, and 76 in the bitmap sequences 610 are sampled to positions 8 through 15 in the sparse bitmap sequences 620. The homozygosity values in position 39 in the bitmap sequences 610 are carried to position 8 in the sparse bitmap sequences 620. Hence, the lower sequence has the value 1. Likewise, the homozygosity values in position 39 in the bitmap sequences 610 are carried to position 9 in the sparse bitmap sequences 620. Hence, the upper sequence has the value 1. In position 46 in the bitmap sequences 610, the homozygosity values are zero for both sequences. As such, the values are zero at position 10 in the sparse bitmap sequences 620.

In some embodiments, the bitmap sequences 610 may be sampled in any suitable manner, arbitrarily or patterned, evenly spaced or not. For example, in some embodiments, the selection of the positions to be sampled to form the sparse bitmap sequences 620 may be completely arbitrary. In other embodiments, some rules may be introduced in the selection of positions in addition to or in alternative to random sampling. For example, the computing server 130 may review a large collection of genetic datasets (e.g., over a million datasets in some embodiments) to determine what positions are likely to contain homozygous mismatches. These positions may be selected by the computing server 130 as key positions to sample. Other rules may also be used in sampling. For example, certain gene locations or SNP locations are known to be related to certain phenotypes (e.g., certain traits of a person or certain diseases). Those positions may also be selected for the sampling process. Alternatively, or additionally, the computing server 130 may choose the SNPs that will go into the sparse bitmaps based on empirical data and properties of those SNPs. By way of example, the computing server 130 chooses the SNPs that have the highest minor allele homozygosity rate from a large sample of data. The computing server 130 may also choose the SNPs based on a minor allele frequency, genotype or imputation error frequency or other criteria.

The computing server 130 may compare the pair of sparse target bitmap sequences to other pairs of sparse bitmap sequences as a pre-scan to eliminate mismatches. The pair of sparse target bitmap sequences may correspond to an encoded and sampled genetic dataset of the target individual. Other pairs of sparse bitmap sequences may correspond to encoded and sampled genetic datasets of other individuals. In some embodiments, since the sparse bitmap sequences are shorter than the full bitmap sequences, comparing sparse bitmap sequences to identify mismatches such as homozygous mismatches can be significantly more efficient than using the full bitmap sequences.

By way of example, if two individuals' genotype data have a homozygous mismatch in the sparse bitmap sequences, the two individuals will also have a homozygous mismatch in the full bitmap sequences or the full genotype datasets. As such, this subset can act as a filter to eliminate seed matches from consideration by comparing fewer data than with the full bitmap sequences.

### Distributed Computing System Architecture

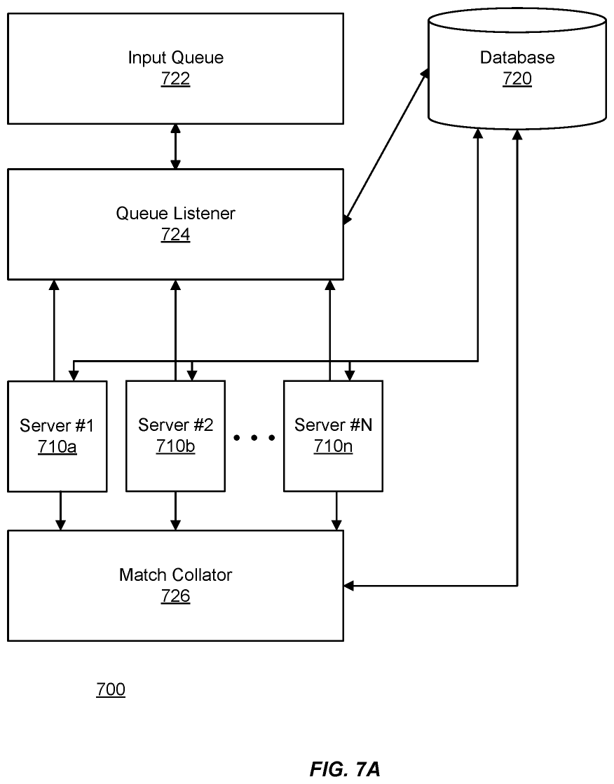

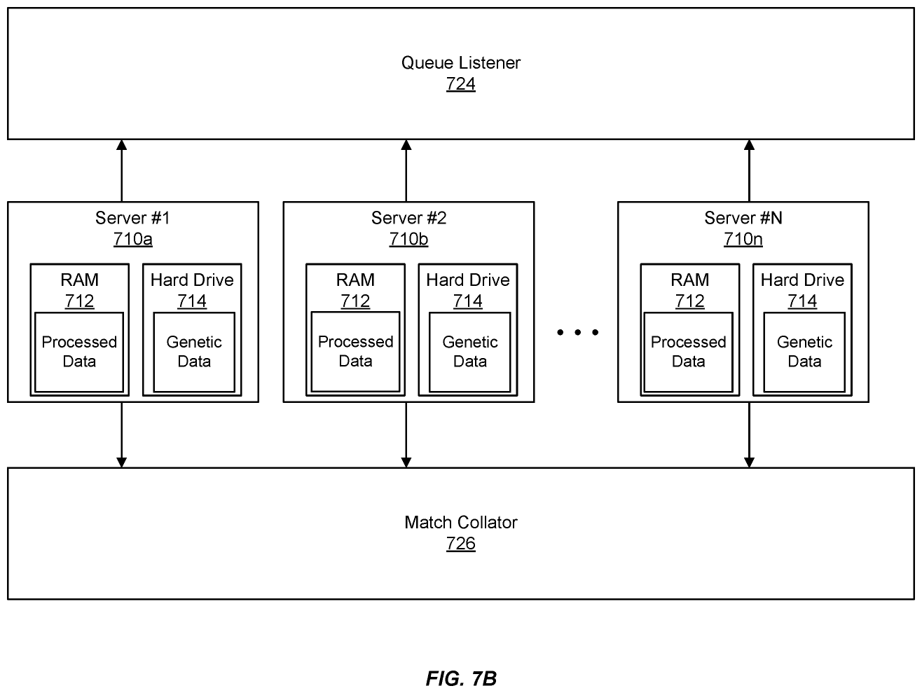

FIG. 7A illustrates the system architecture of a distributed computing system that may be used to carry out the IBD estimation process, in accordance with some embodiments. The system architecture depicted allows efficient determination of IBD relationships between an individual and a large number of individuals. In some embodiments, the architecture in FIG. 7A may represent the structure of IBD estimation engine 225. In some embodiments, the architecture shown in FIG. 7A is a sub-system of the computing server 130. As depicted, multiple servers 710a, 710b, 710n (collectively servers 710 or individually server 710) are in communication with a database 720, which stores genetic data of a plurality of individuals. The servers 710 retrieve genetic data from the database 720 and store such information, process the genetic data such as by encoding the genetic data, and store the processed genetic data in RAM or other memory devices that are different and faster than read and write persistent storage devices—like hard drives—for data access and computation. The genetic data of the target individual is compared to those stored in the RAM in parallel on multiple servers to increase efficiency.

IBD data for a large database is expensive to store in RAM and calculating IBD requires a lot of time loading data. The architecture shown is used to compute IBD between an individual and a large database quickly. This method pre-loads data in RAM of each computing server so that IBD can be computed quickly without requiring that it be stored. Calculating IBD relationships often requires that the computing server 130 spends time reading genotype data of various individuals (e.g., over a million individuals) in a large database. This makes a response often rather slow. Also, even solutions that take advantage of data structures designed for IBD to use database queries assume excellent phasing quality. In some embodiments, the process carried out by the architecture shown in FIG. 7A does not need to assume excellent phasing quality.

In some embodiments, using the architecture shown in FIG. 7A, the IBD computation method may be the same as batch IBD computation (e.g., J-GERMLINE), but the genotype data are stored in hash tables kept in memory of running processes distributed across several servers. A query can distribute genotype data for a query individual to those servers where the computation is performed, and the results (output) delivered to an endpoint where it can be collated and returned. In some embodiments, the IBD computation method is an encoding method illustrated in FIGS. 6 and 7 and encoded bitmaps may be saved in RAM of different servers.

By way of example, the system 700 may include several servers, an input queue 722, a queue listener 724, an output match collator 726, and a database 720. When the system 700 starts, each server 710 loads a portion of the database 720 (e.g., each server may be responsible for an equal share of the database) and creates the data structures (e.g., hash tables, bitmaps) in memory. Each server then waits for tasks that are triggered by input queues. When a query (including genotype data or information on where to find them) comes into the input queue 722, the queue listener 724 distributes the genotype data to each server 710. The queue listener 724 may also be referred to as a data distributor or an ID translator. For the queue listener 724, phased genotype data and Timber weights for the query instance are distributed to each server 710 directly or via the database 720. Mapping between identifiers of Timber and J-GERMLINE, which may be two systems that use different identifiers, may be handled and a translation of data to formalize the data in these two systems may also be carried out.

In some embodiments, the hash tables store haplotypes for a specific window of the genome. The hash tables may be indexed by haplotype. For example, the hash table is a collection of key-value pairs and the keys are haplotypes. The value for each key-value pair may be a collection of individuals who have the haplotypes and other related data. As such, if a new query person has the same haplotype as one of the individuals stored on that server, it is fast to identify their shared haplotypes. The hash table may be used in the same way as it is for J-GERMLINE. In FIG. 4, the haplotypes highlighted would be grouped together and therefore efficient to pair up without comparing all the DNA of individuals that do not share identical DNA with a query individual.

Referring back to FIG. 7A, each server 710 computes on the query data and the partial database it has loaded. Each server 710 may perform IBD determination and relationship estimation for the data that it has loaded. Individual servers 710 may connect directly to the database 720 or may receive input from the queue listener 724. Each server 710 writes information about IBD to the output (match) collator 726. When all servers have responded, the output (match) collator 726 returns the results, possibly by writing information to the database 720, or by transferring it to an output queue or a data endpoint.

Various disclosed embodiments are advantageous over other systems in that IBD can be computed for one individual (or a few individuals) in only the time necessary for computation (often less than a second for a database that typically takes orders of magnitude more time to load from a disk).

The servers in various embodiments, such as servers 710, may be physical servers that are operated by the computing server 130. In some embodiments, the servers may also be distributed servers in the Cloud that are provided by various Cloud server providers, such as AMAZON AWS, GOOGLE, etc.

FIG. 7B is a block diagram illustrating an example version of the system of FIG. 7A, where memories of each server are depicted, as according to some embodiments. Each server stored, in the RAM 712, processed data, which may be processed data in batch IBD computation (e.g., J-GERMLINE) such as hash tables, encoded data such as the encoded bitmaps illustrated in FIGS. 5 and 6, both, or any suitable processed data. The servers 710 each hold a portion of the database and data structures (e.g., hash tables) in RAM 712. The portions of the database could be grouped based on a shared quality between individuals in the database, such as by using reference panels of individuals.

Each server may hold much larger genotype data for their portion of the database in the hard drive 714. The queue listener 724 receives a query related to a target individual and distributes the genetic data of the target individual to each server 710. Phased genotype data and Timber weights are distributed directly or via the database 720 (not shown in FIG. 7B). In some embodiments, for IBD determination that involves the encoding algorithm, the encoded bitmaps of different individuals may be distributed among various servers. A target individual's genetic data may be encoded, or processed, and input into multiple servers for matching against the processed data stored in the RAM 712 of each server 710. The servers 710 compare the query data and the portion of the database it has loaded in the RAM 712 and can perform IBD estimation and relationship estimation. The servers 710 each respond, and the output (match) collator 726 returns results concerning the target, query individual. The results are used to determine the relationship between the target individual and the portion of individuals stored in a server 710. The servers 710 may sustain the encoded data of a portion of a database in the RAM, to be used in reiterating the IBD estimation process with subsequent target, or query, individuals. Sustaining the encoded data allows the system 700 to be much more efficient than having to re-encode data for every target individual.

The distributed architecture is significantly faster than other data processing hardware structure. Without the distributed architecture described in various embodiments, the genetic data of a large number of individuals are in a database that uses hard drives 714 as the underlying storage hardware. When an IBD comparison for a new target individual is to be performed, the computing server needs to retrieve the genetic data from hard drives 714, process the genetic data (e.g., performing phasing, creating hash table, loading parameters, and/or encoding data), and perform the IBD comparison. Since the data size is large given the large number of individuals who have genetic data stored in the computing server while the RAM is limited for a particular server, conventionally the computing server without the distributed architecture cannot save all processed data of every individual in RAM. As such, to complete the process of comparing the genetic data of a target individual with a universe of individuals' genetic data, the computing server without the distributed architecture would need to retrieve a batch of genetic data from a hard drive, process the batch, store the batch in RAM, perform the comparison, delete the batch, and then retrieve another batch and repeat the process. As a second target individual's genetic data comes in at a later time, the computing server without the distributed architecture may need to repeat the whole process because data are constantly retrieved and erased from the RAM due to the sheer volume of data stored in the computing server.

In contrast, the distributed architecture provides a significantly improved computing speed for IBD comparison. Genetic data of various individuals may be distributed among multiple servers so that the processed data may be brought down to a manageable size for each server to handle. The server may store the processed data in RAM 712 and sustain the processed data in RAM 712 for a significantly longer duration. As such, when a second target individual's genetic data comes in at a later time, the computing server 130 may simply use the processed data stored in the RAM to perform the IBD comparison without having to re-process genetic data of various individuals stored in the computing server 130.

In some embodiments, the prolonged storage of the processed data in RAM is novel. It can be expensive to hold such a large set of processed data in memory. In some embodiments, new users genetic data may be added to the computing server 130 and processed daily, the computing server 130 may more efficient to hold the processed data in RAM for a prolonged period instead of writing the data to persistent storage and then reading data from that storage location into RAM.

In some embodiments, the IBD comparison may also be used for other purposes such as determining one or more genetic communities to which a target individual belongs. For example, the processed genetic data stored in various servers may correspond to processed genetic data of reference panels of various genetic communities. In some embodiments, each server may store the processed genetic data of reference panels of a particular genetic community in its corresponding RAM. The target individual's genetic data may be compared by each server to the data of the reference panels to quickly identify one or more genetic communities to which the target individual may belong.

### Computing Machine Architecture

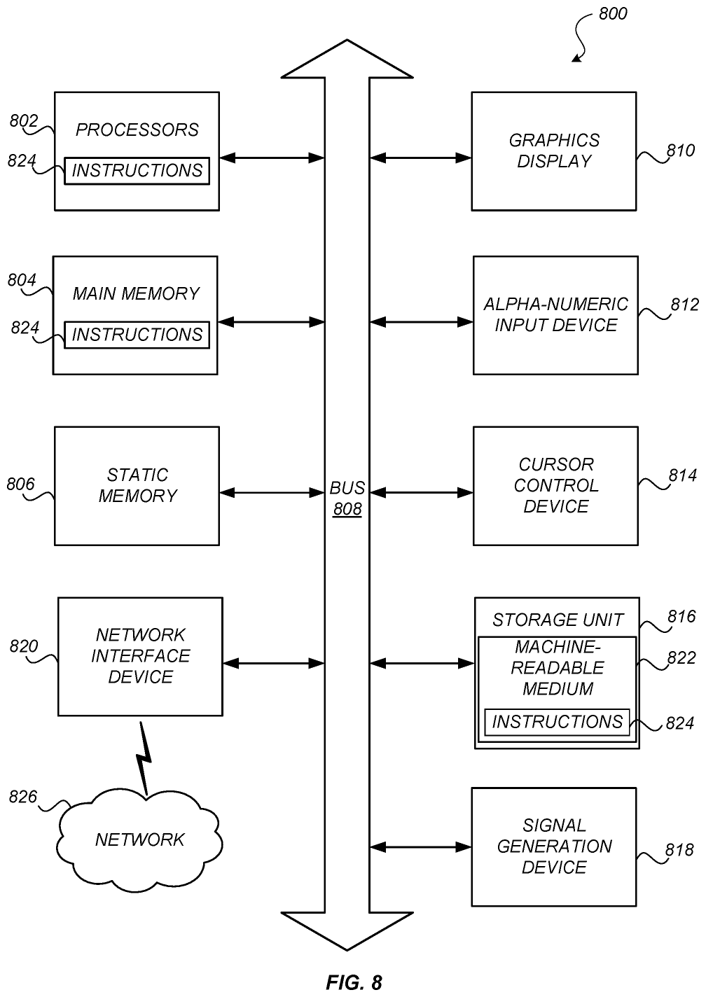

FIG. 8 is a block diagram illustrating components of an example computing machine that is capable of reading instructions from a computer-readable medium and execute them in a processor (or controller). A computer described herein may include a single computing machine shown in FIG. 8, a virtual machine, a distributed computing system that includes multiple nodes of computing machines shown in FIG. 8, or any other suitable arrangement of computing devices.

By way of example, FIG. 8 shows a diagrammatic representation of a computing machine in the example form of a computer system 800 within which instructions 824 (e.g., software, source code, program code, expanded code, object code, assembly code, or machine code), which may be stored in a computer-readable medium for causing the machine to perform any one or more of the processes discussed herein may be executed. In some embodiments, the computing machine operates as a standalone device or may be connected (e.g., networked) to other machines. In a networked deployment, the machine may operate in the capacity of a server machine or a client machine in a server-client network environment, or as a peer machine in a peer-to-peer (or distributed) network environment.

The structure of a computing machine described in FIG. 8 may correspond to any software, hardware, or combined components shown in FIGS. 1 and 2, including but not limited to, the client device 110, the computing server 130, and various engines, interfaces, terminals, and machines shown in FIG. 2. While FIG. 8 shows various hardware and software elements, each of the components described in FIGS. 1 and 2 may include additional or fewer elements.

By way of example, a computing machine may be a personal computer (PC), a tablet PC, a set-top box (STB), a personal digital assistant (PDA), a cellular telephone, a smartphone, a web appliance, a network router, an internet of things (IoT) device, a switch or bridge, or any machine capable of executing instructions 824 that specify actions to be taken by that machine. Further, while only a single machine is illustrated, the term “machine” and “computer” may also be taken to include any collection of machines that individually or jointly execute instructions 824 to perform any one or more of the methodologies discussed herein.

The example computer system 800 includes one or more processors 802 such as a CPU (central processing unit), a GPU (graphics processing unit), a TPU (tensor processing unit), a DSP (digital signal processor), a system on a chip (SOC), a controller, a state equipment, an application-specific integrated circuit (ASIC), a field-programmable gate array (FPGA), or any combination of these. Parts of the computing system 800 may also include a memory 804 that store computer code including instructions 824 that may cause the processors 802 to perform certain actions when the instructions are executed, directly or indirectly by the processors 802. Instructions can be any directions, commands, or orders that may be stored in different forms, such as equipment-readable instructions, programming instructions including source code, and other communication signals and orders. Instructions may be used in a general sense and are not limited to machine-readable codes. One or more steps in various processes described may be performed by passing through instructions to one or more multiply-accumulate (MAC) units of the processors.

One and more methods described herein improve the operation speed of the processors 802 and reduces the space required for the memory 804. For example, the database processing techniques and machine learning methods described herein reduce the complexity of the computation of the processors 802 by applying one or more novel techniques that simplify the steps in training, reaching convergence, and generating results of the processors 802. The algorithms described herein also reduces the size of the models and datasets to reduce the storage space requirement for memory 804.

The performance of certain operations may be distributed among more than one processor, not only residing within a single machine, but deployed across a number of machines. In some example embodiments, the one or more processors or processor-implemented modules may be located in a single geographic location (e.g., within a home environment, an office environment, or a server farm). In other example embodiments, one or more processors or processor-implemented modules may be distributed across a number of geographic locations. Even though in the specification or the claims may refer some processes to be performed by a processor, this should be construed to include a joint operation of multiple distributed processors.

The computer system 800 may include a main memory 804, and a static memory 806, which are configured to communicate with each other via a bus 808. The memory 804 may take the form of random-access memory (RAM). The computer system 800 may further include a graphics display unit 810 (e.g., a plasma display panel (PDP), a liquid crystal display (LCD), a projector, or a cathode ray tube (CRT)). The graphics display unit 810, controlled by the processors 802, displays a graphical user interface (GUI) to display one or more results and data generated by the processes described herein. The computer system 800 may also include alphanumeric input device 812 (e.g., a keyboard), a cursor control device 814 (e.g., a mouse, a trackball, a joystick, a motion sensor, or other pointing instruments), a storage unit 816 (a hard drive, a solid-state drive, a hybrid drive, a memory disk, etc.), a signal generation device 818 (e.g., a speaker), and a network interface device 820, which also are configured to communicate via the bus 808.

The storage unit 816 includes a computer-readable medium 822 on which is stored instructions 824 embodying any one or more of the methodologies or functions described herein. The instructions 824 may also reside, completely or at least partially, within the main memory 804 or within the processor 802 (e.g., within a processor's cache memory) during execution thereof by the computer system 800, the main memory 804 and the processor 802 also constituting computer-readable media. The instructions 824 may be transmitted or received over a network 826 via the network interface device 820.

While computer-readable medium 822 is shown in an example embodiment to be a single medium, the term “computer-readable medium” should be taken to include a single medium or multiple media (e.g., a centralized or distributed database, or associated caches and servers) able to store instructions (e.g., instructions 824). The computer-readable medium may include any medium that is capable of storing instructions (e.g., instructions 824) for execution by the processors (e.g., processors 802) and that cause the processors to perform any one or more of the methodologies disclosed herein. The computer-readable medium may include, but not be limited to, data repositories in the form of solid-state memories, optical media, and magnetic media. The computer-readable medium does not include a transitory medium such as a propagating signal or a carrier wave.

### Additional Considerations

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Any feature mentioned in one claim category, e.g. method, can be claimed in another claim category, e.g. computer program product, system, storage medium, as well. The dependencies or references back in the attached claims are chosen for formal reasons only. However, any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject matter may include not only the combinations of features as set out in the disclosed embodiments but also any other combination of features from different embodiments. Various features mentioned in the different embodiments can be combined with explicit mentioning of such combination or arrangement in an example embodiment or without any explicit mentioning. Furthermore, any of the embodiments and features described or depicted herein may be claimed in a separate claim and/or in any combination with any embodiment or feature described or depicted herein or with any of the features.

Some portions of this description describe the embodiments in terms of algorithms and symbolic representations of operations on information. These operations and algorithmic descriptions, while described functionally, computationally, or logically, are understood to be implemented by computer programs or equivalent electrical circuits, microcode, or the like. Furthermore, it has also proven convenient at times, to refer to these arrangements of operations as engines, without loss of generality. The described operations and their associated engines may be embodied in software, firmware, hardware, or any combinations thereof.

Any of the steps, operations, or processes described herein may be performed or implemented with one or more hardware or software engines, alone or in combination with other devices. In some embodiments, a software engine is implemented with a computer program product comprising a computer-readable medium containing computer program code, which can be executed by a computer processor for performing any or all of the steps, operations, or processes described. The term “steps” does not mandate or imply a particular order. For example, while this disclosure may describe a process that includes multiple steps sequentially with arrows present in a flowchart, the steps in the process do not need to be performed in the specific order claimed or described in the disclosure. Some steps may be performed before others even though the other steps are claimed or described first in this disclosure. Likewise, any use of (i), (ii), (iii), etc., or (a), (b), (c), etc. in the specification or in the claims, unless specified, is used to better enumerate items or steps and also does not mandate a particular order.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein. In addition, the term “each” used in the specification and claims does not imply that every or all elements in a group need to fit the description associated with the term “each.” For example, “each member is associated with element A” does not imply that all members are associated with an element A. Instead, the term “each” only implies that a member (of some of the members), in a singular form, is associated with an element A. In claims, the use of a singular form of a noun may imply at least one element even though a plural form is not used.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights.

The following applications are incorporated by reference in their entirety for all purposes: (1) U.S. Pat. No. 10,679,729, entitled “Haplotype Phasing Models,” granted on Jun. 9, 2020, (2) U.S. Pat. No. 10,223,498, entitled “Discovering Population Structure from Patterns of Identity-By-Descent,” granted on Mar. 5, 2019, (3) U.S. Pat. No. 10,720,229, entitled “Reducing Error in Predicted Genetic Relationships,” granted on Jul. 21, 2020, (4) U.S. Pat. No. 10,558,930, entitled “Local Genetic Ethnicity Determination System,” granted on Feb. 11, 2020, (5) U.S. Pat. No. 10,114,922, entitled “Identifying Ancestral Relationships Using a Continuous Stream of Input,” granted on Oct. 30, 2018, (6) U.S. Patent Publication Application No., entitled “Linking Individual Datasets to a Database,” US2021/0216556, published on Jul. 15, 2021, (7) U.S. Pat. No. 10,692,587, entitled “Global Ancestry Determination System,” granted on Jun. 23, 2020, and (8) U.S. Patent Application Publication No. US 2021/0034647, entitled “Clustering of Matched Segments to Determine Linkage of Dataset in a Database,” published on Feb. 4, 2021.

What is claimed is:

1. A computer-implemented method, comprising:
storing genetic datasets of a plurality of individuals on one or more hard drives of a database;
encoding the genetic datasets of the plurality of individuals to generate pairs of encoded bitmap sequences based on an encoding scheme, wherein the genetic dataset of each individual is encoded to generate a pair of encoded bitmap sequences, the encoding scheme defining a sequence of values based on homozygosity of the genetic dataset of each individual;
storing the pairs of encoded bitmap sequences in random-access memory (RAM) of a plurality of servers, each server's RAM storing the pairs of encoded bitmap sequences of a subset of individuals of the plurality of individuals, wherein the RAM is volatile and has a faster processing speed than the one or more hard drives;
receiving an input pair of encoded bitmap sequences of a target individual for determining relationships between the target individual and the plurality of individuals;
determining matched segments between the target individual and the plurality of individuals, wherein determining matched segments comprises comparing the input pair of the target individual to the pairs that are stored in the RAM, wherein each server operates in parallel with other servers for comparisons of different individuals;
computing, for each server, a relationship between the target individual and an individual of the plurality of individuals;
collating the computed relationships; and
sustaining the pairs of encoded bitmap sequences of the plurality of individuals in the RAM of the plurality of servers.

2. The computer-implemented method of claim 1, wherein the genetic datasets comprise phased genotype datasets or genotype datasets.

3. The computer-implemented method of claim 1, wherein the encoded bitmap sequences of the subset of individuals stored in one of the servers comprises the encoded bitmap sequences of a reference panel of a genetic community.

4. The computer-implemented method of claim 3, further comprising:
determining that the target individual belongs to the genetic community;
selecting the one of the servers that stores the encoded bitmap sequences of the reference panel;
using the one of the servers to determine relationships between the target individual and the reference panel.

5. The computer-implemented method of claim 1, further comprising, for at least one server, creating a hash table.

6. The computer-implemented method of claim 5, further comprising sustaining encoded data in the hash tables and storing:
a hash of a user's account name,
a hash of a user's name,
a hash of a user's date of birth,
a hash of a user's location of birth, or
a hash of a combination of a user's information.

7. The computer-implemented method of claim 1, wherein determining matched segments between the target individual and the plurality of individuals comprise:
comparing encoded data of the target individual that encodes a first type of homogeneous locations of the target individual to a second encoded data encoding a second type of homogeneous locations of another dataset; and
identifying a common location that indicates the encoded data of the target individual and the other dataset in comparison are both homogeneous.

8. The computer-implemented method of claim 1, wherein the encoding scheme of the pairs of encoded bitmap sequences defines that a first encoded bitmap sequence has a first value if a pair of data value sequences are homogenous of a first type and has a second value otherwise, and the encoding scheme defines that a second encoded target bitmap sequence has the first value if the pair of data value sequences are homogeneous of the second type and has the second value otherwise.

9. The computer-implemented method of claim 1, wherein the relationships between the target individual and the plurality of individuals correspond to identity by descent (IBD) relationships.

10. The computer-implemented method of claim 1, wherein the pairs of encoded bitmap sequences of the plurality of individuals are sustained in the RAM of the plurality of servers for a user-defined length of time or until power off of a server.

11. The computer-implemented method of claim 1, further comprising:
receiving a second input pair of encoded bitmap sequences of a second target individual;
comparing the second input pair of encode bitmap sequences to the pairs of encoded bitmap sequences of the plurality of individuals that are sustained in the RAM of the plurality of servers.

12. The computer-implemented method of claim 10, wherein the pairs of encoded bitmap sequences of the plurality of individuals are sustained in the RAM of the plurality of servers for comparisons for a plurality of targeted individuals without regenerating the pairs of encoded bitmap sequences from the genetic datasets stored on the one or more hard drives.

13. A non-transitory computer-readable storage medium comprising instructions executable by a processor, the instructions when executed causing the processor to perform actions comprising:
storing genetic datasets of a plurality of individuals on one or more hard drives of a database;
encoding the genetic datasets of the plurality of individuals to generate pairs of encoded bitmap sequences based on an encoding scheme, wherein the genetic dataset of each individual is encoded to generate a pair of encoded bitmap sequences, the encoding scheme defining a sequence of values based on homozygosity of the genetic dataset of each individual;
storing the pairs of encoded bitmap sequences in random-access memory (RAM) of a plurality of servers, each server's RAM storing the pairs of encoded bitmap sequences of a subset of individuals of the plurality of individuals, wherein the RAM is volatile and has a faster processing speed than the one or more hard drives;
receiving an input pair of encoded bitmap sequences of a target individual for determining relationships between the target individual and the plurality of individuals;
determining matched segments between the target individual and the plurality of individuals, wherein determining matched segments comprises comparing the input pair of the target individual to the pairs that are stored in the RAM, wherein each server operates in parallel with other servers for comparisons of different individuals;
computing, for each server, a relationship between the target individual and an individual of the plurality of individuals; collating the computed relationships; and sustaining the pairs of encoded bitmap sequences of the plurality of individuals in the RAM of the plurality of servers.

14. The non-transitory computer-readable storage medium of claim 13, wherein the genetic datasets comprise phased genotype datasets or genotype datasets.

15. The non-transitory computer-readable storage medium of claim 13, wherein the encoded bitmap sequences of the subset of individuals stored in one of the servers comprises the encoded bitmap sequences of a reference panel of a genetic community.

16. The non-transitory computer-readable storage medium of claim 15, further comprising:
determining that the target individual belongs to the genetic community;
selecting the one of the servers that stores the encoded bitmap sequences of the reference panel;
using the one of the servers to determine relationships between the target individual and the reference panel.

17. The non-transitory computer-readable storage medium of claim 13, wherein determining matched segments between the target individual and the plurality of individuals comprise:
comparing encoded data of the target individual that encodes a first type of homogeneous locations of the target individual to a second encoded data encoding a second type of homogeneous locations of another dataset; and
identifying a common location that indicates the encoded data of the target individual and the other dataset in comparison are both homogeneous.

18. The non-transitory computer-readable storage medium of claim 13, wherein the encoding scheme of the pairs of encoded bitmap sequences defines that a first encoded bitmap sequence has a first value if a pair of data value sequences are homogenous of a first type and has a second value otherwise, and the encoding scheme defines that a second encoded target bitmap sequence has the first value if the pair of data value sequences are homogeneous of the second type and has the second value otherwise.

19. A system comprising:
a database comprising one or more hard drives configured to store genetic datasets of a plurality of individuals; and
a plurality of servers in communication with the database, wherein the plurality of servers are configured to:
encode the genetic datasets of the plurality of individuals to generate pairs of encoded bitmap sequences based on an encoding scheme, wherein the genetic dataset of each individual is encoded to generate a pair of encoded bitmap sequences, the encoding scheme defining a sequence of values based on homozygosity of the genetic dataset of each individual;
store the pairs of encoded bitmap sequences in random-access memory (RAM) of the plurality of servers, each server's RAM storing the pairs of encoded bitmap sequences of a subset of individuals of the plurality of individuals, wherein the RAM is volatile and has a faster processing speed than the one or more hard drives;
receive an input pair of encoded bitmap sequences of a target individual for determining relationships between the target individual and the plurality of individuals;
determine matched segments between the target individual and the plurality of individuals, wherein determining matched segments comprises comparing the input pair of the target individual to the pairs that are stored in the RAM, wherein each server operates in parallel with other servers for comparisons of different individuals;
computing, for each server, a relationship between the target individual and an individual of the plurality of individuals; and
sustain the pairs of encoded bitmap sequences of the plurality of individuals in the RAM of the plurality of servers.

20. The system of claim 19, wherein the encoded bitmap sequences of the subset of individuals stored in one of the servers comprises the encoded bitmap sequences of a reference panel of a genetic community.
